(19) United States
(12) Patent Application Publication
(10) Pub. No.: US 2025/0108177 A1
(43) Pub. Date: Apr. 3, 2025

(54) Electronic system, user interface member, drug delivery device and method for detecting whether a drug delivery device is, or was, exposed to fluid

(71) Applicant: Sanofi, PARIS (FR)

(72) Inventors: Paul Richard Draper, Warwick, Warwickshire (GB); Anthony Paul Morris, Warwick, Warwickshire (GB); Samuel Keir Steel, Warwick, Warwickshire (GB)

(21) Appl. No.: 18/694,113

(22) PCT Filed: Sep. 22, 2022
(86) PCT No.: PCT/EP2022/076296

§ 371 Date: Mar. 21, 2024

(30) Foreign Application Priority Data
Sep. 24, 2021 (EP) 21315184.8

(51) Int. Cl. A61M 5/50

(52) U.S. Cl. CPC A61M 5/5086

(57) ABSTRACT

Electronic system, user interface member, drug delivery device and method for detecting whether a drug delivery device is, or was, exposed to fluid In at least one embodiment, the electronic system is configured to compare measurements taken with a measurement (21) unit with a reference and to determine, based on the result of the comparison, an exposure of the drug delivery device to fluid. The measurements are suitable to provide information about the drug delivery device.

### TECHNICAL FIELD

An electronic system for a drug delivery device is provided. Furthermore, a user interface member for a drug delivery device, a drug delivery device and a method for detecting whether a drug delivery device is, or was, exposed to fluid is provided.

### BACKGROUND

Administering an injection is a process which presents a number of risks and challenges for users and healthcare professionals, both mental and physical. A drug delivery device may aim to make self-injection easier for patients. Drug delivery devices using electronics are becoming increasingly popular in the pharmaceutical industry as well as for users or patients. A stable operation of the electronic components is desirable in order to guarantee that the drug delivery device operates correctly.

### SUMMARY

One object to be achieved is to provide an improved electronic system for a drug delivery device. Preferably, the electronic system may allow to detect an exposure of an electric element of the drug delivery device to fluid. A further object to be achieved is to provide an improved user interface member, an improved assembly, and an improved drug delivery device and an improved method for detecting whether a drug delivery device is, or was, exposed to fluid.

These objects are achieved, inter alia, by the subject matter of the independent claims. Advantageous embodiments and further developments are subject of the dependent claims and are also presented in the following description and in the figures.

Firstly, the electronic system for a drug delivery device is specified.

According to at least one embodiment, the electronic system is configured to compare measurements taken with a measurement unit with a reference. The measurement unit may be part of the electronic system. Particularly, the electronic system may be configured to determine a deviation of the measurements taken with the measurement unit from the reference. Each measurement may be associated with a measurement value or reading of the measurement unit, respectively. The measurement values may be in form of analogue signals of the measurement unit or digitized signals of the measurement unit. For example, the analogue measurement values are converted with help of an Analogue-to-Digital Converter (ADC) to digital signals. The reference may be a reference value or a distribution of reference values or a curve of reference values, respectively.

Here and in the following, comparing the measurements with the reference may particularly mean comparing the respective measurement values with the reference value(s), e.g. by determining a difference between the measurement values and the reference value(s) and/or by determining whether the measurement values exceed the reference value(s) or are below the reference value(s).

According to at least one embodiment, comparing the measurements with the reference comprises comparing an amplitude of the measurements with an amplitude of the reference.

According to at least one embodiment, comparing the measurements with the reference comprises comparing a dynamic range of the measurements with a dynamic range of the reference.

According to at least one embodiment, comparing the measurements with the reference comprises comparing a curve shape of the measurements with a curve shape of the reference. This comparison may comprise determining a chi²-value between the curve of the measurements and the curve of the reference.

The measurement unit may be or may comprise a sensor. The measurement unit may, in particular, be an electric or electronic measurement unit, which provides measurements in form of electric or electronic signals and/or which needs electrical power to be operated.

According to at least one embodiment, the measurements are suitable to provide information about the drug delivery device. In other words, the information about the drug delivery device is extractable from the measurements. The information may be, e.g., information on a state of the drug delivery device or an operation process performed by or with the drug delivery device. The information on the state of the device may be information which is different from the information whether the drug delivery device or an element thereof is, or was, exposed to fluid.

Nevertheless, in the present disclosure, the measurements may be evaluated to check whether the drug delivery device or an element thereof is, or was, exposed to fluid.

For example, the measurement unit is configured to measure or detect, respectively, an operation process of the drug delivery device. The operation process may be a drug delivery process or a dose setting process or an activation process of the drug delivery device. The measurements or the measurement values, respectively, may be indicative of the operation process. For example, the measurement unit is configured to detect a movement of an element of the drug delivery device relative to the measurement unit and the measurements may be indicative of such a movement.

According to at least one embodiment, the electronic system is configured to determine an exposure of the drug delivery device or the electronic system to fluid based on the result of the comparison. Particularly, this means that it can determine whether the drug delivery device, e.g. an interior of the drug delivery device or a part in the interior, in particular an electric element of the drug delivery device, is, or was, exposed to fluid. Determining whether such an exposure has happened is done based on the result of the comparison. The fluid may be a gaseous or a liquid fluid, e.g. water or moisture.

For example, if the amplitude and/or the dynamic range and/or the curve shape of the measurements deviates from the amplitude and/or the dynamic range and/or the curve shape of the reference, e.g. by more than a predetermined threshold, it may be determined that the drug delivery device is or was exposed to fluid. Otherwise it may be determined that the drug delivery device was not yet exposed to fluid

For example, if the measurement values or a maximum, minimum or average measurement value of the measurement values exceed(s) or lies below the reference value or a maximum, minimum or average reference value, e.g. by a predetermined threshold, it is determined that the drug delivery device is, or was, exposed to fluid. Otherwise it may be determined that the drug delivery device was not yet exposed to fluid.

In at least one embodiment, the electronic system for a drug delivery device is configured to compare measurements taken with the measurement unit with a reference and to determine, based on the result of the comparison, an exposure of the drug delivery device to fluid. The measurements are suitable to provide information about the drug delivery device.

The present invention is, inter alia, based on the recognition that moisture or water or other fluids may have several effects on a drug delivery device, e.g. on a system thereof which detects the dialing and/or dispensing of doses. If the drug delivery device utilizes an optical encoder system, for example, readings or measurements from the measurement unit(s) in form of optical sensor(s) may be influenced by the presence of moisture, water or other fluids due to a number of effects.

A water droplet may disturb the passage of light from and/or to the optical sensor, causing refraction or magnification of the intended light path. Water droplets near to the optical sensor may contact the electrical tracks which provide power to the active emitter side of the optical sensor, or detect voltage/current on the receiver side of the optical sensor, introducing a change of resistance, capacitance or inductance between individual tracks. Such disturbance of the optical light path, or moisture contacting the electrical tracks leading to the optical sensor, may be detected by monitoring of the sensor readings. Indeed, the inventors observed that empirical data captured during the development build phases show that sensor readings or measurements can be influenced by the presence of water. Water may cause sensor readings or measurements to increase towards a saturation level, which is not normally reached during standard operation. Therefore, a deviation of the readings of the sensor from an expected value (reference) can be used to determine that there is, or was, moisture influencing the functionality of the sensor.

With the electric system specified herein, it may be possible to detect the presence of fluid before the fluid has any harmful effect on the use or robustness of the drug delivery device. In such instances, the user could be alerted to the presence of fluid, and, e.g., advised that device performance could be compromised.

According to at least one embodiment, the measurement unit is configured to be arranged in the drug delivery device or is arranged in the drug delivery device. Particularly, the measurement unit is configured to be arranged in an interior of the drug delivery device or of a member of the drug delivery device.

According to at least one embodiment, the measurements used for comparing with a reference are measurements taken during a measurement period. The measurement period may be a predetermined period of time, e.g. of several seconds. For example, the measurement period is at least 0.1 second or at least 0.5 seconds or at least 1 second or at least 10 seconds. Additionally or alternatively, the measurement period may be at most 50 seconds or at most 20 seconds or at most 15 seconds. During the measurement period, the measurement unit may take a plurality of measurements, e.g. at least 10 measurements or at least 100 or at least 1000 measurements and/or at most 10000 measurements. The measurements may be taken periodically during a measurement period, e.g. with a frequency of at least 100 Hz or at least 1000 Hz and/or at most 5000 Hz. For example, the frequency is between 500 Hz and 4000 Hz. Outside of measurement periods, the measurement unit may not take measurements, e.g., it may be switched off.

When comparing the measurements with the reference, only measurements of one measurement period may be considered. For example, the maximum measurement value of at least some or all of the measurements of the measurement period or the average measurement value of at least some or all of the measurements of the measurement period may be compared to the reference.

According to at least one embodiment, the electronic system is configured to repeat, e.g. periodically repeat, the comparison of measurements with a reference and to repeatedly determine, based on the result of the comparison, an exposure of the drug delivery device to fluid. For example, the electronic system is configured to compare the measurements of a measurement period with a reference after this measurement period is finished and before the next measurement period starts.

According to at least one embodiment, the measurements are measurements of periodically repeated measurement periods. For example, the measurement periods are repeated at least every minute or at least every hour or at least every day, e.g. independently of whether the drug delivery device is used or not.

According to at least one embodiment, the electronic system is configured to adjust the reference to a change in the operational voltage of the measurement unit. For example, a change of the operational voltage in form of a voltage drop appears during aging of the drug delivery device or results from a depletion effect due to usage. The electronic system may be configured to adjust the reference to this voltage drop.

The measurement unit may be powered by a power source, like a battery. Such a power source may show an aging or depletion effect in form of decreasing voltage provided by the power source (operational voltage). A decreasing operational voltage, in turn, influences the measurements of the measurement unit. For example, the maximum reading of the measurement unit decreases with decreasing voltage. Such a decreasing voltage is taken into account by adjusting the reference, e.g., by making the reference time-dependent. For example, the reference is set to decrease with increasing time. The adjustment of the reference may be predetermined, e.g., by manually setting different references for different points in time after the first use of the device. This setting may be done during manufacturing of the drug delivery device and/or during programming of the electronic system.

According to at least one embodiment, the electronic system is configured to adjust the reference based on voltage data wherein the voltage data are indicative for the operational voltage of the measurement unit. In this case, the electronic system is particularly configured to dynamically adjust the reference, namely based on the voltage data.

According to at least one embodiment, the electronic system is configured to adjust the reference based on previous measurements of the measurement unit taken during a preceding period of time. In this case, the electronic system is particularly configured to dynamically adjust the reference based on previous measurements.

For example, the reference is adjusted to follow a trend in the previous measurements of the measurement unit. For example, if the previous measurements, e.g. the amplitude or the maximum measurement value, have increased or decreased with time during the preceding period of time, the reference is also increased or decreased. The reference may also be chosen as to be previous measurements or an average of previous measurements.

According to at least one embodiment, the reference is a fixed reference. In this case, the reference is not changed with time. For example, the reference is determined based on empirical data gathered during development of the drug delivery device.

According to at least one embodiment, the drug delivery device is configured to perform several drug delivery processes one after the other. For example, during each such drug delivery process, a dialled dose is delivered to a user. The drug delivery device may be configured to perform at least 10 or at least 100 or at least 1000 drug delivery processes. It may be necessary to change a drug container storing a drug for performing so many drug delivery processes.

According to at least one embodiment, the electronic system is configured to adjust the reference based on the measurements of the measurement unit associated with the last n drug delivery processes, where n is greater than or equal to 1. For example, n is at least 5 or at least 10. Additionally or alternatively, n is at most 50 or at most 20.

Measurements being associated with a drug delivery process are in particular measurements taken during the drug delivery process or shortly before or after the drug delivery process, e.g., within a time window of at most one minute before and/or after the start or the end of the drug delivery process, respectively.

For example, the maximum or average measurement values associated with the different drug delivery processes are determined for each drug delivery process and the reference value is then adjusted based on the last n maximum or average measurement values. For example, the reference value is set to the average of the last n maximum or average measurement values.

According to at least one embodiment, the measurements are measurements taken during usage of the drug delivery device, particularly during a drug delivery process, i.e. during the period when the drug is actually delivered. For example, the measurement period is the period of the drug delivery process.

According to at least one embodiment, the measurements are measurements taken during a time period when the drug delivery device is not used, e.g. during a time period between two subsequent drug delivery processes. Particularly, the measurements may be taken shortly before or after a drug delivery process. The measurement period may then be a time period between two subsequent drug delivery processes.

According to at least one embodiment, measurements at the beginning of a measurement period are not used (e.g. discounted or discarded) for comparing with the reference. For example, at least the first 5 or at least the first 10 or at least the first 15 measurements of the measurement period are not used. Alternatively or additionally, the electronic system may be configured such that one or more measurements during an initial phase of the measurement period are not compared with the reference. That is to say, these measurements may be disregarded for the comparison operation. The initial phase may comprise the first couple of measurements, e.g. 5 or more or 10 or more or 15 or more, taken and/or be of a duration of more than 2 ms. Only measurements obtained after the initial phase has ended, e.g. from the 16th measurement or the 17th measurement, may be considered for the comparison.

The operational voltage of the measurement unit at the beginning of the measurement period, e.g. at the beginning of a delivery process, may decrease, e.g. during the first measurements. This decrease produces a rapid reduction in the associated measurement values. After the first measurements, the voltage may stabilize to a constant operational voltage for the remainder of the measurement period. Hence, disregarding the initial measurements for the comparison is advantageous as, if they were considered, they might incorrectly suggest a fluid ingress into the system.

According to at least one embodiment, the measurement unit is a sensor, e.g. of the electronic system or the drug delivery device. The sensor may be configured to measure the amount of the delivered dose during a drug delivery process. The sensor may, in particular, be configured to detect a relative movement, especially a relative rotation, between the sensor and a further element, e.g. a movable element of the drug delivery device.

In other words: the measurements may be suitable for providing information about an amount of a dose set during a dose setting process or are indicative for an amount of a delivered dose during a dose delivery process.

According to at least one embodiment, the measurement unit is an optical sensor. The optical sensor may be configured to emit radiation and detect a portion of the radiation reflected by a movable member of the drug delivery device. The optical sensor may comprise an LED, e.g. an infrared LED, and a sensor element configured to detect a reflected portion, e.g. reflected from movable element, of the radiation emitted by the LED. The movable member may comprise alternating areas of different reflectivities for the radiation emitted by the optical sensor. That is to say, the movable member may comprise an encoder structure. The movable member may be an encoder member or an encoder component. The areas of different reflectivities may be areas of different color, e.g. black and white areas.

According to at least one embodiment, the electronic system is configured to produce an output signal in order to communicate to a user if an exposure of the drug delivery device to fluid is determined. The output signal is, in particular, produced only if an exposure of the drug delivery device to fluids has been determined.

According to at least one embodiment, the electronic system comprises at least one processor or is a processor. Particularly, the processor may be configured to receive the measurements of the measurement unit and/or to compare the measurements with the reference and/or to determine whether there is, or was, an exposure of the drug delivery device to fluid based on the result of the comparison. The processor may also be configured to produce the output signal.

According to at least one embodiment, the electronic system further comprises the measurement unit for taking the measurements.

According to at least one embodiment, the electronic system comprises a communication unit. The communication unit may be configured for communicating to a user if an exposure of the drug delivery device to fluid is determined. For example, the communication unit is operated based on the output signal when an exposure of the drug delivery device to fluid is determined.

The communication unit may be an LED configured to emit light, e.g. white light. The drug delivery device may be configured such that the light emitted by the LED may be visible to a user using the drug delivery device.

Additionally or alternatively, the drug delivery device may comprise a communication unit in the form of an acoustic sound generator, e.g. a loudspeaker, configured to provide an acoustic signal in order to communicate the determined exposure to fluid.

The communication of the possible exposure to fluid to the user may indicate to the user that the drug delivery device could provide wrong results or should no longer be used.

According to at least one embodiment, the electronic system comprises a circuit board, e.g., a PCB, such as a flexi-rigid PCB (PCB: printed circuit board). The measurement unit and/or the processor and/or the communication unit(s) may be arranged on the circuit board and/or may be electrically connected to the circuit board.

The electronic system may further comprise a battery for powering the processor and/or the measurement unit and/or the communication unit(s). Moreover, the electronic system may comprise a wireless communication unit, e.g. a Bluetooth unit, e.g. for communicating information obtained with help of the measurement unit to an external device, like a smart phone or computer. For example, a delivered dose measured with help of the measurement unit is communicated to the external device.

The electronic system may also comprise an Analogue-to-Digital Converter, e.g. to convert analog signals form the measurement unit to digital signals. The electronic system may also comprise an electro-mechanical switch, e.g. for activating and/or deactivating a powering of the measurement unit.

According to at least one embodiment, the electronic system is configured to determine an exposure of the drug delivery device to fluid based on the measurements of the measurement unit and measurements of a further measurement unit. Particularly, the drug delivery device and/or the electronic system may comprise a further measurement unit. All features disclosed in connection with the measurement unit are also disclosed for the further measurement unit and vice versa. Particularly, the further measurement unit may be a sensor, like an optical sensor, particularly for measuring the amount of the delivered dose during a delivery process. The sensors of the measurement unit and the further measurement unit may be out of phase with respect to the encoder structure provided by the movable member (see further above). The sensors and the encoder structure may be adjusted such that the sensor outputs (i.e. electrical signals), in combination, during relative movement (e.g. relative rotation) between the sensors and the encoder structure are suitable to provide a multi-bit Gray-Code, e.g. a 2-bit Gray-Code. The combination of the sensor outputs may be unique for a number of different relative positions between the movable member and the sensor(s). A 2-bit Gray-Code characterizes four relative positions uniquely, for example.

For example, the electronic system is configured to determine an exposure of the drug delivery device to fluid only if the measurements of each of the measurement units indicates an exposure to fluid. For example, only if the comparison of measurements of both measurement units with a respective reference individually indicates an exposure to fluid, an exposure of the drug delivery device to fluid is determined. The determination whether the further measurement unit indicates an exposure of the drug delivery device to fluid may be done in the same way as for the measurement unit, e.g. when measurement values of the further measurement unit exceed a reference value.

Next, the user interface member is specified. The user interface member may be a knob or a button permanently connected or connectable to a container holder of the drug delivery device or releasably connectable to the container holder of the drug delivery device.

According to at least one embodiment, the user interface member comprises an electronic system as specified above. Therefore, all features disclosed in connection with the electronic system are also disclosed for the user interface member.

The electronic system may be arranged in an interior of the user interface member. For example, the electronic system may be circumferentially surrounded by a housing element of the user interface member. The electronic system may be arranged inside the user interface member so that it is protected from fluid.

According to at least one embodiment, the user interface member is configured to be touched by the user in order to operate the user interface member for performing a dose dial and/or a drug delivery process. Thus, the user interface member may be configured to perform a dose dial and/or drug delivery process when connected to the container holder and when operated by the user.

For example, the user interface member comprises a lateral surface which forms an outer surface of the component and which is configured to be grabbed by a user. The lateral surface may delimit the user interface member in outward radial direction. Particularly, the lateral surface may run parallel or acute-angled to a longitudinal axis of the user interface member or drug delivery device.

The lateral surface may be configured to be grabbed by a user using two fingers for performing a rotation of the user interface member around the longitudinal axis, e.g. with respect to the container holder. Additionally or alternatively, the user interface member may comprise a proximal surface facing in proximal direction. The proximal surface may run perpendicularly or obliquely with respect to the longitudinal axis and/or the lateral surface. The proximal surface may be configured to be touched by a user, e.g. using only one finger, particularly for pushing the user interface member in a distal direction.

According to at least one embodiment, the lateral surface may comprise gripping features, e.g. grooves. The grooves may extend parallel or acute-angled to the longitudinal axis. The gripping features may simplify grabbing of the user interface member by a user.

According to at least one embodiment, the user interface member comprises the measurement unit. For example, the measurement unit is arranged in the interior of the user interface member.

Next, the assembly is specified. The assembly may be an assembly for a drug delivery device. The assembly may be attachable to a drug delivery device. Alternatively, the assembly may be part of a drug delivery device or the drug delivery device.

The assembly may comprise a movable member and a measurement unit as described above in the context of the electronic system. Alternatively, or additionally, the assembly may comprise the electronic system as described above.

In one embodiment, the assembly may comprise an electronic system with an optical sensor and an encoder structure, e.g. a Gray-Code encoder ring. The electronic system, e.g. the optical sensor thereof, may be configured to detect and/or quantify a movement of the encoder structure. The movement of the encoder structure may be indicative of a dose dialed with and/or a dose expelled from the drug delivery device, e.g. indicative of the size of the dose dialed or the dose expelled. In one embodiment, the electronic system of the assembly may be configured to detect an ingress of fluid into the assembly or the drug delivery device through a detection of optical, e.g. reflective and/or refractive, properties of fluid droplets present between the optical sensor and the encoder structure, e.g. the Gray-Code encoder ring. If fluid is present, the fluid leads to a change in the output signal of said sensor, e.g. a higher or lower output signal, compared to a situation without exposure to fluid. In other words, the optical sensor may be configured to detect the presence of fluid in the light path between the sensor and the encoder structure by detecting an amount or a portion of radiation arriving at the sensor. In one embodiment, the radiation may be emitted by the optical sensor or a component thereof, e.g. an LED. A portion of this radiation may be refracted by the fluid in the light path before it is reflected by the encoder structure. Alternatively, or additionally, the reflected portion may be refracted again by the fluid in the light path, before arriving at the sensor. The sensor may be configured to detect the arriving portion of the radiation. Due to a comparison with a reference, the assembly, e.g. the electronic system thereof, may determine the presence of fluid in the light path. In other words, the assembly, e.g. the electronic system thereof, may determine an exposure of an element, e.g. an electric element of the drug delivery device, to fluid based on the result of the comparison.

The assembly may particularly include the electronic system specified above. Therefore, all features disclosed in connection with the electronic system are also disclosed for the assembly and vice versa.

Next, the drug delivery device is specified. The drug delivery device may be an injection device and/or a pen type device, e.g. a dial extension pen. The drug delivery device may be a variable dose device in which the drug dose to be delivered to a user can be variably set. For example, the drug delivery device is a reusable device.

According to at least one embodiment, the drug delivery device comprises an electronic system or a user interface member as specified above. Therefore, all features disclosed for the electronic system and/or for the user interface member are also disclosed for the drug delivery device.

The electronic system may, in particular, be arranged in an interior of the drug delivery device. The electronic system may be arranged such that it is protected from fluid reaching into the drug delivery device. Likewise, the measurement unit(s) may be arranged in the interior of the drug delivery device and may be protected from fluid.

According to at least one embodiment, the drug delivery device comprises a container holder for holding a drug container. The container holder may be a housing of the drug delivery device or may be a separate element connected or connectable to the housing. The container holder may be configured to hold the drug container axially and/or rotationally fixed with respect to the housing of the drug delivery device. Particularly, the container holder may hold the drug container such that the drug container does not move in an axial and/or a rotational direction during a drug delivery process.

According to at least one embodiment, the drug container is filled with a drug.

The drug delivery device and/or the user interface member specified herein may be elongated and/or may comprise a longitudinal axis, e.g. a main extension axis. Additionally or alternatively, the drug delivery device and/or the user interface member may have a rotational symmetry with respect to the longitudinal axis. A direction parallel to the longitudinal axis is herein called an axial direction. By way of example, the drug delivery device and/or the user interface member may be cylindrically shaped.

Furthermore, the drug delivery device may comprise an end, e.g. a longitudinal end, which may be provided to face or to be pressed against a skin region of a human body. This end is herein called the distal end. A drug or medicament may be supplied via the distal end. The opposing end is herein called the proximal end. The proximal end is, during usage, remote from the skin region. The axial direction pointing from the proximal end to the distal end is herein called distal direction. The axial direction pointing from the distal end to the proximal end is herein called proximal direction. A distal end of a member or element or feature of the drug delivery device is herein understood to be the end of the member/element/feature located most distally. Accordingly, the proximal end of a member or element or feature is herein understood to be the end of the element/member/feature located most proximally.

In other words, distally is used herein to specify directions, ends or surfaces which are arranged or are to be arranged to face or point towards a dispensing end of the drug delivery device or components thereof and/or point away from, are to be arranged to face away from or face away from the proximal end. On the other hand, proximal is herein used to specify directions, ends or surfaces which are arranged or are to be arranged to face away from or point away from the dispensing end and/or from the distal end of the drug delivery device or components thereof. The distal end may be the end closest to the dispensing end and/or furthest away from the proximal end and the proximal end may be the end furthest away from the dispensing end. A proximal surface may face away from the distal end and/or towards the proximal end and a distal surface may face towards the distal end and/or away from the proximal end. The dispensing end may be a needle end where a needle unit is or is to be mounted to the device, for example.

A direction perpendicular to the longitudinal axis and/or intersecting with the longitudinal axis is herein called radial direction. An inward radial direction is a radial direction pointing towards the longitudinal axis. An outward radial direction is a radial direction pointing away from the longitudinal axis. The term “angular direction”, “azimuthal direction” or “rotational direction” are herein used as synonyms. Such a direction is a direction perpendicular to the longitudinal axis and perpendicular to the radial direction.

A method for operating the drug delivery device may be as follows. A user interface member in the form of a knob connected to the container holder and comprising the electronic system is grabbed by a user, e.g. at the lateral surface of the user interface member, and is rotated thereby dialling a dose to be injected into the user. The knob may be rotated on a helical path with respect to the drug container holder, thereby moving, e.g., in a proximal direction. After having dialled the desired dose, the knob may be pushed in an axial direction, e.g. in the distal direction, and the dose of the drug is injected. For this purpose, the user may press against the proximal surface of the knob. During movement of the knob in the distal direction, the knob itself may not rotate but a movable element of the drug delivery device may rotate. The dialled dose may thereby be ejected, e.g. injected into a patient. The sensor(s) in the knob may measure the rotation of the movable element. The measurements of the sensor(s) may be sent to a processor of the electronic system. The processor may determine the delivered dose on the basis of the measurements. This information may be sent to an external device, e.g. with help of a wireless communication unit. The processor may also compare the measurements with a reference and may then determine, based on the result of this comparison, whether the interior of the drug delivery device, particularly the sensor(s) or at least one other electric element, are, or were, exposed to a fluid.

Next, the method for detecting whether a drug delivery device is, or was, exposed to fluid is specified. The method may particularly be performed with the electronic system, the assembly or the drug delivery device, specified above. Therefore, all features disclosed in connection with the electronic system, the assembly or the drug delivery device are also disclosed for the method and vice versa.

According to at least one embodiment, a measurement unit is arranged in the drug delivery device.

According to at least one embodiment, the method comprises a step of receiving measurements taken with the measurement unit. The measurements may be suitable to provide information about the drug delivery device. In other words: the measurements may be suitable for providing information about an amount of a dose set during a dose setting process or are indicative for an amount of a delivered dose during a dose delivery process.

According to at least one embodiment, the method comprises a step of comparing the measurements with a reference.

According to at least one embodiment, the method comprises a step of determining an exposure of the drug delivery device or an electric element thereof to fluid based on the result of the comparison.

Furthermore, a computer program and a computer readable medium are specified. The computer program comprises instructions which, when the program is executed by a computer, cause the computer to carry out the method for detecting whether a drug delivery device is, or was, exposed to fluid. The computer readable medium has stored therein the computer program.

Hereinafter, the electronic system, the user interface member, the drug delivery device and the method described herein will be explained in more detail with reference to drawings on the basis of exemplary embodiments. Same reference signs indicate same elements in the individual figures. However, the size ratios involved are not necessarily to scale, individual elements may rather be illustrated with exaggerated size for better understanding.

### BRIEF DESCRIPTION OF THE DRAWINGS

FIG. 1 shows an exemplary embodiment of a drug delivery device in an exploded view,

FIGS. 2 and 3 show proximal sections of an exemplary embodiment of the drug delivery device in different views.

FIGS. 4 to 8 show measurements from an exemplary embodiment of the drug delivery device.

### EXEMPLARY EMBODIMENTS

In the following, exemplary embodiments will be described with reference to an insulin injection device. The present disclosure is, however, not limited to such application and may equally well be deployed with injection devices that are configured to eject other medicaments or with drug delivery devices in general, preferably pen-type devices and/or injection devices.

Certain exemplary embodiments in this document are illustrated with respect to a drug delivery device in the form of an injection device comprising a user interface member in the form of a knob which realizes an injection button and a dose setting (dialling) member at the same time, e.g. similar to the devices disclosed in WO 2014/033195 A1 or WO 2014/033197 A1. Thus, the knob may be used for initiating and/or performing a dose delivery operation of the drug delivery device and may also be used for initiating and/or performing a dose setting operation. The devices may be of the dial extension type, i.e., their length increases during dose setting. Other injection devices with the same kinematic behaviour of the dial extension during dose setting and dose expelling operational mode are known as, for example, the Kwikpen® or Savvio® device marketed by Eli Lilly and the FlexPen®, FlexTouch® or Novopen® device marketed by Novo Nordisk. An application of the general principles to these devices therefore appears straightforward and further explanations will be omitted. However, the general principles of the present disclosure are not limited to that kinematic behaviour.

Certain other embodiments may be conceived for application to injection devices where there are separate injection button and grip components/dose setting members e.g. the devices disclosed in WO 2004/078239 A1. Thus, the present disclosure also relates to systems with two separate user interface members, e.g., one for the dose setting operation and one for the dose delivery operation. In order to switch between a dose setting configuration of the device and a dose delivery configuration, the user interface member for dose delivery may be moved relative to the user interface member for dose setting.

If one user interface member is provided, the user interface member may be moved distally relative to a housing. In the course of the respective movement, a clutch between two elements of a dose-setting mechanism and a drive mechanism of the device changes its state, e.g. from engaged to released or vice versa. When the clutch, e.g., formed by sets of meshing teeth on the two elements, is engaged, the two elements may be rotationally locked to one another and when the clutch is disengaged or released, one of the elements may be permitted to rotate relative to the other one of the two elements. One of the elements may be a drive element or drive sleeve which engages a plunger rod of the dose-setting and drive mechanism. The drive sleeve may be designed to rotate relative to the housing during dose setting and may be rotationally locked relative to the housing during dose delivery. The engagement between drive sleeve and plunger rod may be a threaded engagement. Thus, as the drive sleeve cannot rotate during dose delivery, axial movement of the drive sleeve relative to the housing will cause the plunger rod to rotate. This rotation may be converted into axial displacement of the plunger rod during the delivery operation by a threaded coupling between plunger rod and housing.

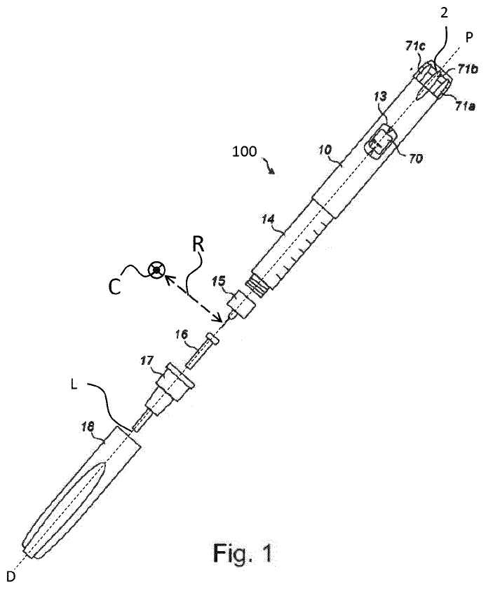

FIG. 1 is an exploded view of an exemplary embodiment of a drug delivery device 100. In this exemplary embodiment, the drug delivery device 100 is an injection device, e.g., a pen-type injector.

The injection device 100 of FIG. 1 is an injection pen that comprises a housing 10 holding a drug container 14, e.g., an insulin container, or a container holder for such a container 14. The container 14 may contain a drug, e.g., insulin. The container 14 may be a cartridge or a receptacle for a cartridge which may contain the cartridge or be configured to receive the cartridge. A needle 15 can be affixed to the container 14 or the receptacle. The container 14 may be a cartridge and the receptacle may be a cartridge holder. The needle 15 is protected by an inner needle cap 16 and either an outer needle cap 17 or another cap 18. An insulin dose to be ejected from the injection device 100 can be set, programmed, or ‘dialled in’ by turning a user interface member 2 in form of a knob 2, and a currently programmed or set dose is then displayed via dose window 13, for instance in multiples of units. The units may be determined by the dose-setting mechanism which may permit relative rotation of the knob 2 to the housing 10 only in whole-number multiples of one unit setting increment, which may define one dose increment. This may be achieved by an appropriate ratchet system, for example. The indicia displayed in the window 13 may be provided on a number sleeve or dial sleeve 70. For example, where the injection device 100 is configured to administer human insulin, the dose may be displayed in so-called International Units (IU), wherein one IU is the biological equivalent of about 45.5 micrograms of pure crystalline insulin ( 1/22 mg). Other units may be employed in injection devices for delivering analogue insulin or other medicaments. It should be noted that the selected dose may equally well be displayed differently than as shown in the dose window 13 in FIG. 1.

The dose window 13 may be in the form of an aperture in the housing 10, which permits a user to view a limited portion of a dial sleeve 70 that is configured to move when the knob 2 is turned, to provide a visual indication of a currently programmed dose. The knob 2 is rotated on a helical path with respect to the housing 10 when turned during programming.

In this exemplary embodiment, the knob 2 includes one or more features 71a, 71b, 71c in form of formations to facilitate gripping and/or attachment of a data collection device or electronic system.

The injection device 100 may be configured so that turning the knob 2 causes a mechanical click sound to provide acoustic feedback to a user. In this embodiment, the knob 2 also acts as an injection button. When needle 15 is stuck into a skin portion of a patient, and then the knob 2 is pushed in an axial direction, the insulin dose displayed in display window 13 will be ejected from injection device 100. When the needle 15 of injection device 100 remains for a certain time in the skin portion after the knob 2 is pushed home, the dose is injected into the patient's body. Ejection of the insulin dose may also cause a mechanical click sound, which is however different from the sounds produced when rotating the knob 2 during dialing of the dose.

In this exemplary embodiment, during delivery of the insulin dose, the knob 2 is returned to its initial position in an axial movement, without rotation, while the dial sleeve 70 or number sleeve 70 is rotated to return to its initial position, e.g. to display a dose of zero units. As noted already, the disclosure is not restricted to insulin but should encompass all drugs in the drug container 14, especially liquid drugs or drug formulations.

The injection device 100 may be used for several injection processes until either the insulin container 14 is empty or the expiration date of the medicament in the injection device 100 (e.g. 28 days after the first use) is reached.

Furthermore, before using injection device 100 for the first time, it may be necessary to perform a so-called “prime shot” to ensure fluid is flowing correctly from insulin container 14 and needle 15, for instance by selecting two units of insulin and pressing knob 2 while holding the injection device 100 with the needle 15 upwards. For simplicity of presentation, in the following, it will be assumed that the ejected amounts substantially correspond to the injected doses, so that, for instance the amount of medicament ejected from the injection device 100 is equal to the dose received by the user.

As explained above, the knob 2 also functions as an injection button so that the same component is used for dialling/setting the dose and dispensing/delivering the dose. Again, we note that a configuration with two different user interface members which, preferably only in a limited fashion, are movable relative to one another is also possible. The following discussion will, however, focus on a single user interface member which provides dose setting and dose delivery functionality. In other words, a setting surface of the member which is touched by the user for the dose setting operation and a dose delivery surface which is touched by the user for the dose delivery operation are immovably connected. Alternatively, they may be movable relative to one another, in case different user interface members are used. During the respective operation, the user interface member is preferably moved relative to the body or housing of the device. During dose setting, the user interface member is moved proximally and/or rotates relative to the housing. During dose delivery, the user interface member moves axially, e.g., distally, preferably without rotating relative to the housing or body.

FIG. 1 also indicates the coordinate system used herein for specifying positions of members or elements or features. The distal direction D and proximal direction P run parallel to the longitudinal axis L. The longitudinal axis L is a main extension axis of the device 100. The radial direction R is a direction perpendicular to the longitudinal axis L and intersecting with the longitudinal axis L. The azimuthal direction C, also referred to as angular direction or rotational direction, is a direction perpendicular to the radial direction R and to the longitudinal axis L. The different directions and axes will not be indicated in every of the following figures in order to increase the clarity of the figures.

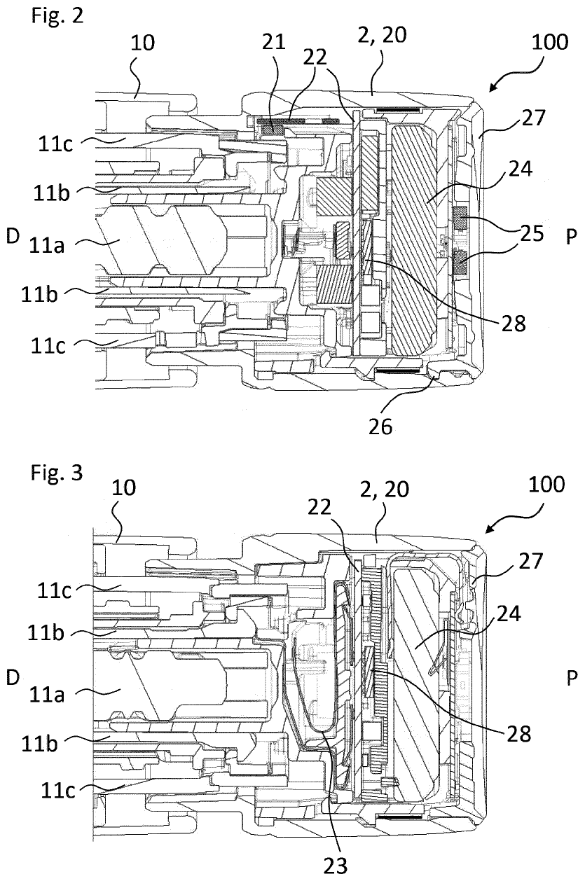

FIG. 2 shows a proximal section of the drug delivery device 100 of FIG. 1 in a cross-sectional view. FIG. 3 shows the same proximal section but in a different cross-sectional view. As can be seen, the user interface member 2 in form of the knob 2 is connected to the housing 10. The knob 2 may be permanently or releasable connected to the housing 10.

The drug delivery device 100 also comprises a plunger rod 11a, a drive sleeve 11b and the dial sleeve 11c. These elements are operatively coupled for the dose dial and dose injection. During dose injection, the dial sleeve 11c rotates together with the number sleeve 70, for example. This rotation is detected by a measurement unit 21 in form of an optical sensor 21 in the interior of the knob 2 which does not rotate during dose injection. For example, the dial sleeve 11c and/or the number sleeve 70 may comprise alternating black and white areas which have different reflectivities for radiation, e.g. radiation emitted by the optical sensor 21. The areas may form an encoder structure. It is also possible to have a surface profile with alternating depressions and elevations in the dial or number sleeve as encoder structure. Particularly, the optical sensor 21 is sensitive to fluid and/or the measurements of the sensor 21 might be affected by the presence of fluid. Although the optical sensor 21 is surrounded by housing elements 20, 27 in form of a grip element 20 and a proximal cover element 27 which are permanently connected by a snap connection 26, it may still happen that fluid reaches to the optical sensor 21. In this case, the measurements of the optical sensor 21 may not be reliable anymore. It would be useful to know whether one can rely on the measurements or not.

In this exemplary embodiment, the user interface member 2 comprises an electronic system having a processor 28. The electronic system is configured to compare measurements of the optical sensor 21 with a reference and to determine, based on the result of this comparison, whether the interior of the user interface member 2, particularly the optical sensor 21, is, or was, exposed to fluid, like water or moisture. The electronic system may also be configured to adjust the reference to a change of the operational voltage of the optical sensor 21 and/or to adjust the reference based on previous measurements of the optical sensor 21 taken during a preceding period of time. For example, the electronic system is configured to adjust the reference based on measurements of the optical sensor 21 associated with the last 5 or last 10 drug delivery processes, where n≥1. All these steps may be performed by the processor 28.

The optical sensor 21 may also be part of the electronic system. The optical sensor 21 and the processor 28 are mounted on a common circuit board 22, e.g. on the same side thereof or on different, e.g. opposite, sides. Measurements of the optical sensor 21 may be sent to the processor 28 via the circuit board 22. The optical sensor may detect radiation reflected from the dial sleeve or number sleeve (which may act as a moving, e.g. rotating, encoder component) onto the sensor. Thus, the sensor expediently comprises a radiation sensitive element, e.g. an optoelectronic detector chip. The radiation, e.g., infrared radiation, may be generated by a radiation source, e.g., a source of the sensor, and radiated towards the encoder component. The radiation can be reflected by the encoder component towards the sensor and excite the radiation sensitive element thereof to produce a signal. Due to the encoder structure provided by the encoder component, e.g. a surface profile with alternating elevations and depressions or alternating areas of different reflectivity such as black and white areas, the amount of radiation (the intensity) reaching the sensor will vary which is detectable via the signals obtained from the sensor. This enables the system to quantify how far the encoder component has rotated relative to the sensor 21. The encoder component may rotate relative to the sensor only during dose delivery (dose injection). Hence, via the amount of the relative rotation the dose of drug which has been delivered during a dose delivery operation can be calculated.

The knob 2 comprises, besides the optical sensor 21, an electro-mechanical switch 23, a battery 24 and LEDs 25. The circuit board 22 may be electrically connected to the optical sensor 21 in order to receive the measurements of the sensor 21 and/or to provide the sensor 21 with electric power. The battery 24 may be used to power the sensor 21, the LEDs 25, the processor 28 and/or further electric or electronic elements on the circuit board 22, like a communication unit (not shown). The mechanical switch 23 may be operated by the drive sleeve 11b when the knob 2 is axially, e.g. distally, moved relative to the drive sleeve 11b. Operating the mechanical switch 23 may lead to powering on of the sensor 21 and/or the LEDs 25 and/or the processor 28.

The LEDs 25 may be configured to communicate to a user an operation or operating state of the drug delivery device 100. For this purpose, the cover element 27 forming the proximal surface of the knob 2 may comprise a transparent region through which light of the LEDs 25 can leave the knob 2. The transparent region is, e.g., formed between the lateral surface of the knob 2 and the proximal surface of the knob 2.

The processor 28 may be configured to generate an output signal in case it determines an exposure of the drug delivery device 100 to fluid. This output signal can be used to operate the LEDs 25 in order to communicate to a user the exposure of the drug delivery device 100 to fluid. Alternatively or additionally, an error code may be generated which may be stored in a memory of the system and/or transmitted to another device.

The knob 2 of FIGS. 2 and 3 or in general the electronic system may comprise two optical sensors 21. Only one of these optical sensors 21 is shown in FIG. 2, the other optical sensor 21, is, e.g., hidden behind other elements. The sensors 21 are expediently out-of-phase relative to the encoder structure on the rotating encoder component, e.g. the dial sleeve or number sleeve. However, systems using just one sensor are also possible. The sensors and the encoder structure may be adjusted, such the combined sensor outputs provide a 2-bit Gray-Code characterizing, e.g. four, unique relative positions between the encoder structure and the sensors.

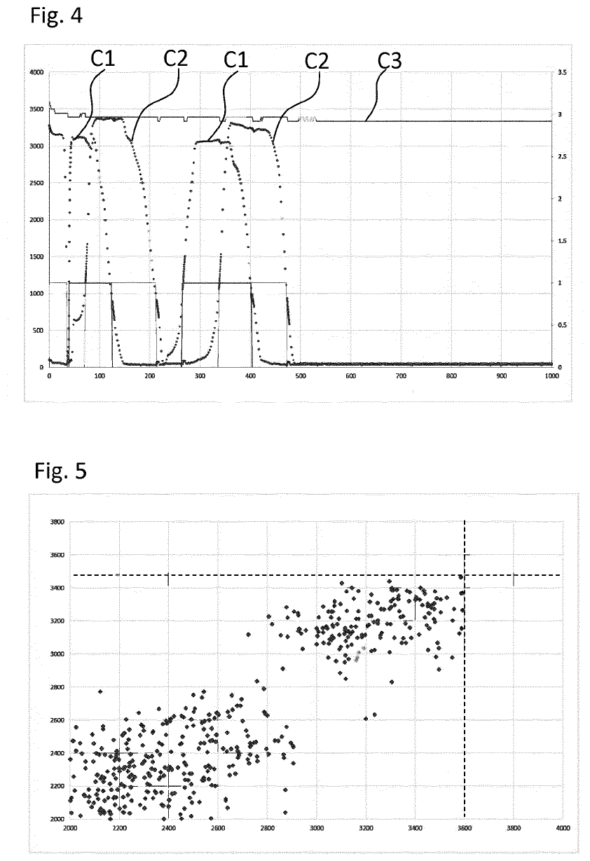

FIG. 4 shows measurements of the optical sensors 21 during normal operation of the sensors 21, i.e. when no fluid has entered into the interior of the knob 2 and has altered the functionality of the sensors 21. The y-axis on the left represents the measurement values after conversion from analogue signals to digital signals in arbitrary units (the ADC may also be part of the electronic system and may be mounted on the circuit board 22). The x-axis represents the time in milliseconds. The curve C1 shows the measurements of the first optical sensor 21, the curve C2 shows the measurements of the second optical sensor 21. The curve C3 shows the operational voltage at the sensors 21 provided by the battery 24 and has the y-axis on the right as its reference, where the unit is, e.g., Volts.

FIG. 4 shows the measurements of one drug delivery process. Each of the curves C1, C2 has peaks and valleys which are associated with the white and black areas (and/or the elevations and depressions) on the dial sleeve and/or number sleeve. E.g., the peaks are associated with the (white) areas for which the reflectivity is high and the valleys are associated with the (black) areas for which the reflectivity is low on the encoder component (dial sleeve and/or number sleeve). For the (white) areas of high reflectivity, the measurement values of the sensors 21 are above 3000. For the (black) areas of low reflectivity, the measurement values are around 0.

Readings or measurements with the (respective) sensor may be obtained or taken with a sampling frequency or rate, e.g. under control of processor 28. That is to say, measurements may be taken at distinct points in time. The sensor may be operated with a sampling frequency or rate of greater than or equal to: 500 Hz 1000 Hz, 2000 Hz, 3000 Hz, 4000 Hz (see also further below), preferably at least when the movement of the encoder component should be monitored, e.g. during the dose delivery operation.

Also visible in FIG. 4 is that the operational voltage drops as time decreases. This voltage drop has as a consequence that also the measurement values will decrease with time. This can already be observed via the difference in the heights of the peaks.

Also visible in FIG. 4 is that, initially, the operating voltage may have irregularities, e.g. between 0 and about 25 on the x-axis. This may be due to the sensor(s) being activated close to the start of the drug delivery process or operation, e.g. via triggering of the switch 23. FIG. 5 shows the respective maximum measurement values of the two sensors 21 for 390 drug delivery processes. The x-axis represents the maximum measurements values of the first optical sensor 21 and the y-axis represents the maximum measurement values for the second optical sensor 21. As can be seen, the maximum measurement values of the first optical sensor 21 are all below 3600, the maximum measurement values of the second optical sensor 21 are all below 3450. These values could be used as the above-mentioned reference values (dashed lines).

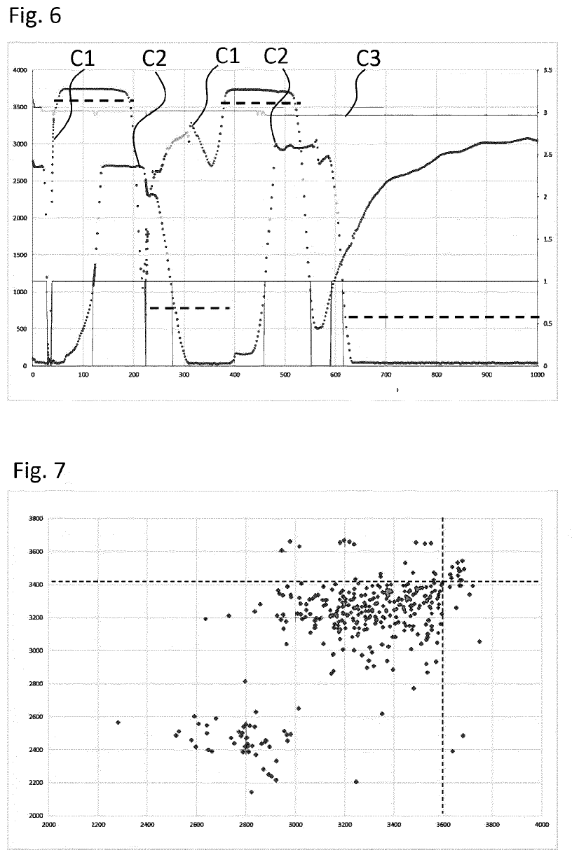

FIG. 6 shows measurements of the optical sensors 21 during a drug delivery process as a function of time when the sensors 21 are exposed to a fluid, like water. As can be seen, also between the peaks, namely when the sensor 21 faces a black area, the measurements are not 0 anymore which is a result of the exposure to fluid. Moreover, the measurement values of the first sensor (curve C1) are considerably higher than in FIG. 4.

In FIG. 6, the dashed horizontal lines indicate reference values which might be used to determine whether there was an exposure to fluid or not, e.g. as sensor readings (measurement values) beyond that are anomalous and suggest liquid ingress into the system. The measurement values of the first sensor 21 exceed these reference values only in FIG. 6 but are well below these reference values in FIG. 4. By comparing the measurement values with the reference values, e.g. by determining that the measurement values or at least a maximum measurement value during a delivery process (measurement period) exceeds the reference values, it may be determined that the sensor was exposed to fluid.

It is possible that one cannot determine whether the respective sensor output or measurement should be in the region of a peak or a valley or at the rising or falling edge or flank of the respective curve during the operation of the system, e.g. during an ongoing dose delivery operation. In this case, only the higher reference values, i.e. the ones indicating that the sensor output or measurement should be in the region of a peak, could be used (in the depicted case these are the values indicated by the first and third dashed line as seen from the left in FIG. 6). Hence, the other values (see ones indicated by the second and fourth dashed lines) may not be suitable as reference values in this case.

Also visible in FIG. 6 is that the reference values (horizontal dashed lines) decrease with increasing time. This takes into account the voltage drop appearing over time as visible in curve C3.

Like FIG. 5, FIG. 7 shows the maximum measurement values of the two sensors 21. However, this time both sensors 21 have been exposed to a fluid. One can see that several maximum measurement values exceed the reference values 3600 and 3450, respectively. The measurements with these maximum measurement values may indicate that the sensors 21 have been exposed to fluid. It is also visible that, for some measurements, both sensors 21 deliver maximum measurement values above the respective reference value. To be more certain that there was indeed an exposure to fluid, only such measurements could be used as an indicator for an exposure.

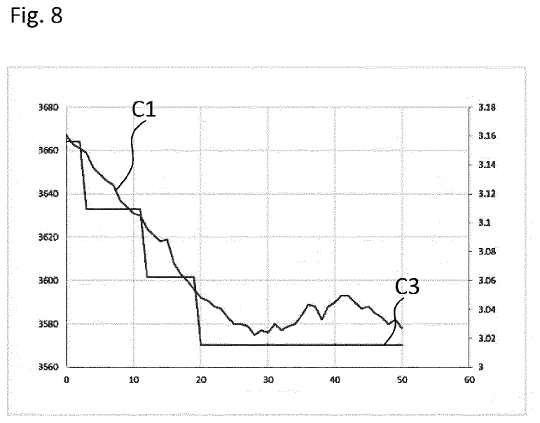

FIG. 8 shows measurements of one of the optical sensors, e.g. the first optical sensor 21, during a drug delivery process (see curve C1). Curve C3 shows the operational voltage applied to the first sensor 21. One can see that, at the beginning of the drug delivery process, the voltage decreases, e.g. comparatively rapidly, (see also the irregularities in FIG. 4 at the beginning of the delivery process which may be the region shown in FIG. 8) and therewith the measurement values of the sensor 21. After some time, the voltage stabilizes and so do the measurement values of the sensor 21. Therefore, for determining whether there was an exposure to fluid, the first measurement values at the beginning of the measurement period (or delivery process) can be omitted. For example, an initial number of measurement values (samples) or measurement values obtained in a predetermined initial time interval since commencement of the dose delivery process or operation may be disregarded and/or may not be compared to the reference values. For example, a number N of measurement values or more may be disregarded where N equals one of the following: 5, 10, 15, 16, 17, 18, 19, 20. Alternatively or additionally, N may be less than or equal to: 30, 25, 20, 19, 18, 17, 16. For example, the initial predetermined time interval may be greater than or equal to: 1 ms, 2 ms, 3 ms, 4 ms. Alternatively or additionally, the initial predetermined time interval may be less than than or equal to: 10 ms, 9 ms, 8 ms, 7 ms, 6 ms, 5 ms, 4 ms. The measurement values during the dose delivery operation or process may be obtained with a (preferably constant) frequency or sampling rate of greater than or equal to one of the following values: 1000 Hz, 1500 Hz, 2000 Hz, 2500 Hz, 3000 Hz, 3500 Hz, 4000 Hz. If the rate is 4000 Hz, for example, the first 16 values (or the first 4 ms of the measurements after the initiation of the dose delivery process) could be disregarded and only measurement values thereafter may be used for comparison with the reference. Initiation of the dose delivery process can be characterized by the switch 23 being triggered, e.g. to produce a switch signal. The switch signal may be indicative that the user presses on knob 2 to deliver a previously set dose. In response to the switch signal, the sensor(s) 21 may be switched on or from a slower response rate to a higher response rate.

During dose delivery processes, the maximum measurement value(s) of the sensor(s) may be determined and/or stored. During or after the process the determined and/or stored values may be compared to the reference value, e.g. 3450. If the maximum measurement value is higher, an error code indicative for fluid ingress into the system can be generated and/or the error could be communicated to the user, e.g. via the LEDs, or another device.

We note that the dynamic range of the measurement values and/or a shape of a curve determined by the measurement values could also be used as reference to determine fluid ingress into or fluid exposure of the system.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir. or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

The invention described herein is not limited by the description in conjunction with the exemplary embodiments. Rather, the invention comprises any new feature as well as any combination of features, particularly including any combination of features in the patent claims, even if said feature or said combination per se is not explicitly stated in the patent claims or exemplary embodiments.

### REFERENCE NUMERALS

10 drug container holder/housing
11a plunger rod
11b drive sleeve
11c dial sleeve
13 dose window
14 drug container
15 needle
16 inner needle cap
17 outer needle cap
18 cap
20 grip element
21 measurement unit/optical sensor
22 circuit board
23 electro-mechanical switch
24 battery
25 LED
26 snap connection
27 cover element
28 processor
70 dial sleeve
71a . . . 71c formation
100 drug delivery device
D distal direction
P proximal direction
L longitudinal axis
R radial direction
C azimuthal direction/rotational direction/angular direction

1. Electronic system for a drug delivery device (100), wherein the electronic system is configured to:
compare measurements taken with a measurement unit (21) with a reference, wherein the measurements are suitable to provide information about the drug delivery device (100),
determine an exposure of the drug delivery device (100) to fluid based on the result of the comparison.

2. Electronic system according to claim 1 configured to determine an exposure of an electric element of the drug delivery device (100) to fluid based on the result of the comparison.

3. Electronic system according to claim 1 or 2, wherein
the electronic system is configured to adjust the reference to a change of the operational voltage of the measurement unit (21) and/or
the electronic system is configured to adjust the reference based on previous measurements of the measurement unit (21) taken during a preceding period of time.

4. Electronic system according to any one of the preceding claims, wherein
comparing the measurements with the reference comprises at least one of: comparing an amplitude of the measurements with an amplitude of the reference, comparing a dynamic range of the measurements with a dynamic range of the reference, comparing a curve shape of the measurements with a curve shape of the reference.

5. Electronic system according to any one of the preceding claims, wherein
the drug delivery device (100) is configured to perform several drug delivery processes one after the other,
the electronic system is configured to adjust the reference based on measurements of the measurement unit (21) associated with the last n drug delivery processes, where n≥1.

6. Electronic system according to any one of the preceding claims, wherein
the measurements are measurements taken during usage of the drug delivery device (100) and/or
the measurements are measurements taken during time periods when the drug delivery device (100) is not used.

7. Electronic system according to any one of the preceding claims, wherein
the measurement unit (21) is a sensor for measuring the amount of the delivered dose during a drug delivery process.

8. Electronic system according to any one of the preceding claims, wherein
the measurement unit (21) is an optical sensor.

9. Electronic system according to any one of the preceding claims, wherein
the electronic system is configured to compare measurements taken with the measurement unit (21) with the reference during a measurement period during which the measurement unit takes measurements, and wherein the electronic system is configured such that one or more measurements during an initial phase of the measurement period are not compared with the reference.

10. Electronic system according to any one of the preceding claims, wherein
the electronic system is configured to produce an output signal in order communicate to a user if an exposure of the drug delivery device (100) to fluid is determined.

11. Electronic system according to any one of the preceding claims, comprising
a processor (27) for receiving the measurements, comparing the measurements with the reference and determining an exposure of the drug delivery device (100) to fluid based on the result of the comparison.

12. Electronic system according to any one of the preceding claims, comprising
the measurement unit (21) for taking the measurements,
a communication unit (25) for communicating to a user if an exposure of the drug delivery (100) device to fluid is determined.

13. Electronic system according to any one of the preceding claims, wherein
the electronic system is configured to determine an exposure of the drug delivery device (100) to fluid based on the measurements of the measurement unit (21) and measurements of a further measurement unit (21).

14. User interface member (2) for a drug delivery device (100),
comprising an electronic system according to any one of the preceding claims,
wherein the user interface member (2) is configured to be touched by a user in order to operate the user interface member for performing a dose dial and/or a drug delivery process.

15. Drug delivery device (100) comprising
an electronic system according to any one of claims 1 to 13 or a user interface member (2) according to claim 14,
a container holder (10) for holding a drug container.

16. Drug delivery device (100) according to claim 15, comprising
a drug container (14) filled with a drug.

17. Method for detecting an exposure of a drug delivery device (100) to fluid, wherein a measurement unit (21) is arranged in the drug delivery device (100), wherein the method comprises:
receiving measurements taken with the measurement unit (21),
comparing the measurements with a reference,
determining an exposure of the drug delivery device (100) to fluid based on the result of the comparison.

18. Method according to claim 17, wherein the measurements taken with the measurement unit (21) are suitable to provide information about the drug delivery device (100).

19. Method according to claim 17 or 18, wherein the method comprises:
determining an exposure of an electric element of the drug delivery device (100) to fluid based on the result of the comparison.

20. Method for according to one of claims 17 to 19, wherein the method is performed by an electronic system according to one of claims 1 to 13.

21. Electronic system for a drug delivery device (100), wherein the electronic system is configured to:
compare measurements taken with a measurement unit (21) with a reference, wherein the measurements are suitable to provide information about the drug delivery device (100),
determine an exposure of an electric element of the drug delivery device (100) to fluid based on the result of the comparison.

22. Electronic system according to one of claim 1 to 13 or 21, wherein the measurement unit (21) is an optical sensor (21) which is configured to emit radiation and detect a portion of the radiation reflected by a movable member of the drug delivery device (100).
